(19) United States
(12) Patent Application Publication
(10) Pub. No.: US 2025/0177389 A1
(43) Pub. Date: Jun. 5, 2025

(54) METHOD OF TREATMENT INCLUDING KRAS G12C INHIBITORS AND SHP2 INHIBITORS

(71) Applicant: ELI LILLY AND COMPANY, Indianapolis, IN (US)

(72) Inventors: Xueqian GONG, Carmel, IN (US); David Michael HYMAN, Westport, CT (US); Xi LIN, Fishers, IN (US); Sheng-Bin PENG, Carmel, IN (US)

(21) Appl. No.: 18/842,544

(22) PCT Filed: Mar. 3, 2023
(86) PCT No.: PCT/US2023/014438

§ 371 Date: Aug. 29, 2024

Related U.S. Application Data
(60) Provisional application No. 63/316,756, filed on Mar. 4, 2022.

(51) Int. Cl. A61K 31/4985; A61K 31/497; A61K 31/553; A61P 35/00

(52) U.S. Cl. CPC A61K 31/4985; A61K 31/497; A61K 31/553; A61P 35/00

(57) ABSTRACT

The present disclosure provides method of treating a patient for cancer, comprising administering to a patient in need thereof, effective amounts of a compound of the formula:

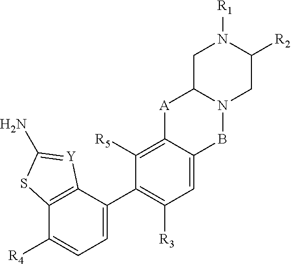

where R₁, R₂, R₃, R₄, R₅, R₆, R₇, R₈, R₉, R₁₀, R₁₁, R₁₂, A, B, and Y are as described herein, or pharmaceutically acceptable salts thereof, and a SHP2 inhibitor, or a pharmaceutically acceptable salt thereof.

The present disclosure relates to a method of treating a patient for cancer, comprising administering to a patient in need thereof, an effective amount of a KRAS G12C inhibitor, or pharmaceutically acceptable salts thereof with a SHP2 inhibitor, or pharmaceutically acceptable salts thereof, to treat cancers such as lung cancer, colorectal cancer, pancreatic cancer, bladder cancer, cervical cancer, endometrial cancer, ovarian cancer, cholangiocarcinoma or esophageal cancer.

Oncogenic KRas mutations have been identified in approximately 30% of human cancers and have been demonstrated to activate multiple downstream signaling pathways. Despite the prevalence of KRas mutations, it has been a difficult therapeutic target. (Cox, A. D. Drugging the Undruggable RAS: Mission Possible? Nat. Rev. Drug Disc. 2014, 13, 828-851; Pylayeva-Gupta, y et al. RAS Oncogenes: Weaving a Tumorigenic Web. Nat. Rev. Cancer 2011, 11, 761-774).

WO2015/054572 and WO2016/164675 disclose certain quinazoline derivatives capable of binding to KRAS G12C. WO2016/044772 also discloses methods of using such quanzoline derivatives. WO2020/0081282 discloses KRAS G12C inhibitors. WO2018/206539 and WO2020/178282 disclose certain heteroaryl compounds capable of binding to KRAS G12C oncoproteins.

SHP2 inhibitors are also known in the art. WO 2019/167000 and WO 2020/022323 disclose certain SHP2 inhibitors.

WO 2018/013597, WO 2019/051084, and US 2020/368238 each disclose certain SHP2 inhibitors in combination with RAS inhibitors.

There remains a need to provide small molecule combinations of KRAS G12C and SHP2 inhibitors. In particular, there is a need to provide more potent, orally deliverable KRAS G12C and SHP2 inhibitors that are useful for treating cancer. More particularly, there is a need to provide combinations of small molecule inhibitors that specifically inhibit KRas GTP and SHP2 activity. There is also a need to provide combinations of small molecule KRAS G12C and SHP2 inhibitors that exhibit synergistic antiproliferative effect and antitumor effect. Further, there is a desire to provide combinations of KRAS G12C and SHP2 inhibitors that overcome bypass of KRAS inhibition treatment. Also, there is a need to provide combinations of KRAS G12C and SHP2 inhibitors that exhibit increased efficacy with reduced or minimized untoward or undesired effects. The present disclosure addresses one or more of these needs by providing combinations and methods and uses for the combinations of KRAS G12C and SHP2 inhibitors.

The present disclosure provides a method of treating a patient for cancer, comprising administering to a patient in need thereof, an effective amount of a compound of Formula I:

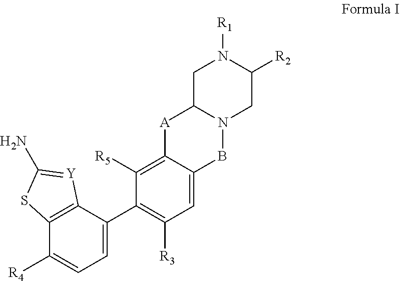

wherein:
   A is —OCH₂—, —N(R₆)CH₂—, —OCH₂CH₂—, —N(R₆)CH₂CH₂—, —CH₂OCH₂—, or —CH₂N(R₆)CH₂—;
   B is —CH₂— or —C(O)—;
   Y is —C(CN)— or —N—;
   R₁ is —CN, —C(O)C—CR₈, or a group of the formula

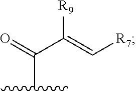

R₂ is H, methyl, or —CH₂CN;
R₃ and R₅ are each independently H, halogen, —C₀₋₃ alkyl-cyclopropyl, —C₁₋₆ alkyl optionally substituted 1-3 times with R₁₀, or —O—C₁₋₆ alkyl optionally substituted 1-3 times with R₁₀;
R₄ is H, halogen, or —C₁₋₆ alkyl optionally substituted 1-3 times with R₁₀;
R₆ is H or —C₁₋₆ alkyl optionally substituted 1-3 times with R₁₀;
R₇ is H, halogen, —NR₁₁R₁₂, —CH₂NR₁₁R₁₂, —C₁₋₆ alkyl optionally substituted 1-3 times with R₁₀ or R₁₃, —C₀₋₃ alkyl cyclopropyl, or —O—C₁₋₆ alkyl optionally substituted 1-3 times with R₁₀ or R₁₃;
R₈ is H, —C₁₋₄ alkyl optionally substituted 1-3 times with R₁₀, or —C₃₋₆ cycloalkyl optionally substituted 1-3 times with R₁₀;
R₉ is H, halogen, —CN, —C₀₋₃ alkyl-C₃₋₆ cycloalkyl, or —C₁₋₆ alkyl optionally substituted 1-3 times with R₁₀;
R₁₀ is independently at each occurrence halogen, oxygen, hydroxy, —C₁₋₄ alkyl, or —O—C₁₋₄ alkyl;
R₁₁ and R₁₂ are each independently H, —C₁₋₄ alkyl, or —C₁₋₄ heteroalkyl, wherein R₁₁ and R₁₂ may combine to form a heterocycloalkyl; and
R₁₃ is independently at each occurrence —N—C₁₋₄ alkyl,
or a pharmaceutically acceptable salt thereof; and
an effective amount of a SHP2 inhibitor, or a pharmaceutically acceptable salt thereof.

As used herein, the term halogen means fluoro (F), chloro (Cl), bromo (Br), or iodo (I). As used herein, the term alkyl means saturated linear or branched-chain monovalent hydrocarbon radicals of one to six carbon atoms, e.g., “—C₁₋₆ alkyl.” Examples of alkyls include, but are not limited to, methyl, ethyl, propyl, 1-propyl, isopropyl, butyl, pentyl, and hexyl. As used herein, the term heteroalkyl means saturated linear or branched-chain monovalent hydrocarbon radicals containing two to five carbon atoms and at least one heteroatom, e.g., “—C₁₋₄ heteroalkyl.” As used herein, the term cycloalkyl means saturated monovalent cyclic molecules with three to six carbon atoms, e.g., “—C₃₋₆ cycloalkyl.” Examples of cycloalkyl groups include, but are not limited to, cyclopropyl, cyclobutyl, cyclopentyl, and cyclohexyl. As used herein, the term cycloheteroalkyl means saturated monovalent cyclic molecules with two to five carbon atoms and at least one heteroatom, e.g., “—C₃₋₆ cycloheteroalkyl.” Examples of cycloheteroalkyl groups include, but are not limited to, pyrrolidine, piperidine, imidazolidine, pyrazolidine, and piperazine.

In cases where a zero is indicated, e.g., —C₀₋₃ alkyl-C₃₋₆ cycloalkyl, the alkyl component of the substituent group can be absent, thus, if R₉ of Formula I is a cyclopropyl group with no lead alkyl, the substituent would be described by the —C₀₋₃ alkyl-cyclopropyl substituent as described for R₉ (i.e., the substituent group would be —C₀— cyclopropyl).

Regarding R₁₁ and R₁₂, the two groups may combine with the nitrogen they are attached to when chemistry allows to form a heterocycloalkyl. Examples of said heterocycloalkyl groups include, but are not limited to, piperidine, piperazine, and morpholine.

In an embodiment the present disclosure provides a method of treating a patient for cancer, comprising administering to a patient in need thereof, an effective amount of a compound of Formula I, or a pharmaceutically acceptable salt thereof, wherein the compound of Formula I is a compound of Formula Ia:

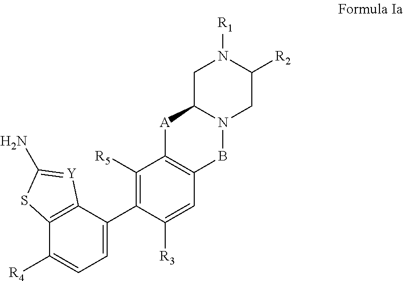

where R₁, R₂, R₃, R₄, R₅, A, B, and Y are as defined above, or a pharmaceutically acceptable salt thereof; and an effective amount of a SHP2 inhibitor, or a pharmaceutically acceptable salt thereof.

In an embodiment A is —OCH₂—, —N(R₆)CH₂—, —OCH₂CH₂—, —N(R₆)CH₂CH₂— in the compound of Formula I or Ia, or a pharmaceutically acceptable salt thereof. In a further embodiment A is —OCH₂— or —OCH₂CH₂— in the compound of Formula I or Ia, or a pharmaceutically acceptable salt thereof. In yet a further embodiment A is —OCH₂CH₂— in the compound of Formula I or Ia, or a pharmaceutically acceptable salt thereof.

In a further embodiment B is —C(O)— in the compound of Formula I or Ia, or a pharmaceutically acceptable salt thereof.

In a further embodiment Y is —C(CN)— in the compound of Formula I or Ia, or a pharmaceutically acceptable salt thereof.

In a further embodiment Y is —N— in the compound of Formula I or Ia, or a pharmaceutically acceptable salt thereof.

In a further embodiment R₁ is —CN, —C(O)C≡CR₈ in the compound of Formula I or Ia, or a pharmaceutically acceptable salt thereof. In yet a further embodiment R₁ is a group of the formula:

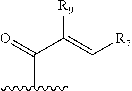

in the compound of Formula I or Ia, or a pharmaceutically acceptable salt thereof.

In a further embodiment R₂ is H or methyl in the compound of Formula I or Ia, or a pharmaceutically acceptable salt thereof. In yet a further embodiment R₂ is H in the compound of Formula I or Ia, or a pharmaceutically acceptable salt thereof.

In a further embodiment R₃ is H, halogen, methyl, methoxy, ethyl, isopropyl, or cyclopropyl in the compound of Formula I or Ia, or a pharmaceutically acceptable salt thereof. In yet a further embodiment R₃ is halogen, (preferably F or Cl) in the compound of Formula I or Ia, or a pharmaceutically acceptable salt thereof.

In a further embodiment R₄ is H or halogen in the compound of Formula I or Ia, or a pharmaceutically acceptable salt thereof. In yet a further embodiment R₄ is H, F, or Cl in the compound of Formula I or Ia, or a pharmaceutically acceptable salt thereof.

In a further embodiment R₅ is halogen (preferably Cl) in the compound of Formula I or Ia, or a pharmaceutically acceptable salt thereof.

In a further embodiment R₆ is H or CH₃ in the compound of Formula I or Ia, or a pharmaceutically acceptable salt thereof.

In a further embodiment R₉ is H, F, Cl, —CH₂F, —CF₃, or —CH₂OH in the compound of Formula I or Ia, or a pharmaceutically acceptable salt thereof. In yet a further embodiment R₉ is H in the compound of Formula I or Ia, or a pharmaceutically acceptable salt thereof.

In a further embodiment R₇ is H, —CH₂, —CH₂F, —CH₂OH, —CH₂OCH₃, —CH₂N(CH), or —CH₂-morpholine in the compound of Formula I or Ia, or a pharmaceutically acceptable salt thereof. In yet a further embodiment R₇ is H in the compound of Formula I or Ia, or a pharmaceutically acceptable salt thereof.

In yet a further embodiment R₉ is H and R₇ is H, —CHF₂, —CH₂F, —CH₂OH, —CH₂OCH₃, —CH₂N(CH₃)₂, or —CH₂-morpholine in the compound of Formula I or Ia, or a pharmaceutically acceptable salt thereof.

In yet a further embodiment R₉ is H, F, Cl, —CH₂F, —CF₃, or —CH₂OH in the compound of Formula I or Ia and R₇ is H in the compound of Formula I or Ia, or a pharmaceutically acceptable salt thereof.

In yet a further embodiment R₇ and R₉ are both H in the compound of Formula I or Ia, or a pharmaceutically acceptable salt thereof.

In yet a further embodiment R₁ is —CN, or —C(O)C—CR₈ in the compound of Formula I or Ia and R₈ is H, methyl, —CH₂F, or —CH₂OH in the compound of Formula I or Ia, or a pharmaceutically acceptable salt thereof.

In yet a further embodiment R₁ is a group of the formula:

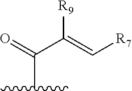

in the compound of Formula I or Ia,
and R₇ and R₉ are both H in the compound of Formula I or Ia, or a pharmaceutically acceptable salt thereof.

In yet a further embodiment R₁ is a group of the formula:

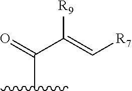

in the compound of Formula I or Ia,
and R₇ is tert-butyl in the compound of Formula I or Ia, and R₉ is —CN in the compound of Formula I or Ia, or a pharmaceutically acceptable salt thereof.

In yet a further embodiment A is —OCH₂—, —N(R₆)CH₂—, —OCH₂CH₂—, —N(R₆)CH₂CH₂— in the compound of Formula I or Ia, and B is —C(O)— in the compound of Formula I or Ia, or a pharmaceutically acceptable salt thereof.

In yet a further embodiment A is —OCH₂— or —OCH₂CH₂— in the compound of Formula I or Ia and B is —C(O)— in the compound of Formula I or Ia, or a pharmaceutically acceptable salt thereof.

In yet a further embodiment A is —OCH₂CH₂— in the compound of Formula I or Ia and B is —C(O)— in the compound of Formula I or Ia, or a pharmaceutically acceptable salt thereof.

In yet a further embodiment A is —OCH₂—, —N(R₆)CH₂—, —OCH₂CH₂—, or —N(R₆)CH₂CH₂— in the compound of Formula I or Ia, B is C(O) in the compound of Formula I or Ia, and R₂ is H or —CH₃ in the compound of Formula I or Ia, or a pharmaceutically acceptable salt thereof.

In yet a further embodiment A is —OCH₂— or —OCH₂CH₂—, B is —C(O)— in the compound of Formula I or Ia, and R₂ is H or methyl, in the compound of Formula I or Ia, or a pharmaceutically acceptable salt thereof.

In yet a further embodiment A is —OCH₂CH₂—, in the compound of Formula I or Ia, B is —C(O)— in the compound of Formula I or Ia, and R₂ is H or methyl, in the compound of Formula I or Ia, or a pharmaceutically acceptable salt thereof.

In yet a further embodiment A is —OCH₂—, —N(R₆)CH₂—, —OCH₂CH₂—, —N(R₆)CH₂CH₂—, in the compound of Formula I or Ia, B is —C(O)—, in the compound of Formula I or Ia, and R₂ is H, in the compound of Formula I or Ia, or a pharmaceutically acceptable salt thereof.

In yet a further embodiment A is —OCH₂— or —OCH₂CH₂—, in the compound of Formula I or Ia, B is —C(O)—, in the compound of Formula I or Ia, and R₂ is H, in the compound of Formula I or Ia, or a pharmaceutically acceptable salt thereof.

In yet a further embodiment A is —OCH₂CH₂—, in the compound of Formula I or Ia, B is —C(O)—, in the compound of Formula I or Ia, and R₂ is H, in the compound of Formula I or Ia, or a pharmaceutically acceptable salt thereof.

In yet a further embodiment A is —OCH₂CH₂—, in the compound of Formula I or Ia, and R₂ is H or methyl, in the compound of Formula I or Ia, or a pharmaceutically acceptable salt thereof.

In yet a further embodiment A is —OCH₂CH₂—, in the compound of Formula I or Ia, and R₂ is H, in the compound of Formula I or Ia, or a pharmaceutically acceptable salt thereof.

In yet a further embodiment B is —C(O)—, in the compound of Formula I or Ia, and R₂ is H or methyl, in the compound of Formula I or Ia, or a pharmaceutically acceptable salt thereof.

In yet a further embodiment B is —C(O)—, in the compound of Formula I or Ia, and R₂ is H, in the compound of Formula I or Ia, or a pharmaceutically acceptable salt thereof.

In yet a further embodiment R₃ and R₅ are each independently selected from H, halogen or methyl, in the compound of Formula I or Ia, or a pharmaceutically acceptable salt thereof.

In yet a further embodiment R₃ or R₅ are halogen, in the compound of Formula I or Ia, or a pharmaceutically acceptable salt thereof.

In yet a further embodiment R₃ and R₅ are halogen, in the compound of Formula I or Ia, or a pharmaceutically acceptable salt thereof.

In yet a further embodiment R₃ and R₅ are each independently selected from F or Cl, in the compound of Formula I or Ia, or a pharmaceutically acceptable salt thereof.

In yet a further embodiment Y is —C(CN)—, in the compound of Formula I or Ia, and R₄ is H or halogen (preferably F or Cl), in the compound of Formula I or Ia, or a pharmaceutically acceptable salt thereof.

In yet a further embodiment Y is —N—, in the compound of Formula I or Ia, and R₄ is H or halogen (preferably F or Cl), in the compound of Formula I or Ia, or a pharmaceutically acceptable salt thereof.

In yet a further embodiment Y is —C(CN)—, in the compound of Formula I or Ia, and R₃ and R₅ are each independently selected from methyl or halogen, in the compound of Formula I or Ia, or a pharmaceutically acceptable salt thereof.

In yet a further embodiment Y is —C(CN)—, in the compound of Formula I or Ia, and R₃ and R₅ are each halogen (preferably F or Cl), in the compound of Formula I or Ia, or a pharmaceutically acceptable salt thereof.

In yet a further embodiment Y is —N—, in the compound of Formula I or Ia, R₃ and R₅ are each independently selected from methyl or halogen, in the compound of Formula I or Ia, or a pharmaceutically acceptable salt thereof.

In yet a further embodiment Y is —N—, in the compound of Formula I or Ia, R₃ and R₅ are each halogen (preferably F or Cl), in the compound of Formula I or Ia, or a pharmaceutically acceptable salt thereof.

In yet a further embodiment A is —OCH₂—, —OCH₂CH₂—, —N(R₆)CH₂CH₂—, —CH₂OCH₂—, or —CH₂N(R₆)CH₂, in the compound of Formula I or Ia; B is —CH₂— or —C(O)—, in the compound of Formula I or Ia; Y is —C(CN)— or —N—, in the compound of Formula I or Ia; R₁ is —CN, —C(O)C—CR₈, or a group of the formula:

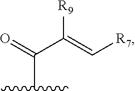

in the compound of Formula I or Ia;
R₂ is H or methyl, in the compound of Formula I or Ia; R₃ and R₅ are each H, F, Cl or methyl, in the compound of Formula I or Ia; R₄ is H or F; R₆ is H or methyl, in the compound of Formula I or Ia; R₇ is H, —CHF₂, —CH₂F, —CH₂OH, —CH₂OCH₃, —CH₂N(CH₃)₂, —CH₂-morpholine or tert-butyl, in the compound of Formula I or Ia; R₈ is methyl, —CH₂F or —CH₂OH, in the compound of Formula I or Ia; and R₉ is H, F, Cl, —CH₂F, —CF₃, —CH₂OH or CN, in the compound of Formula I or Ia; or a pharmaceutically acceptable salt thereof.

In yet a further embodiment A is —OCH₂— or —OCH₂CH₂—, in the compound of Formula I or Ia; B is —CH₂— or —C(O)—, in the compound of Formula I or Ia; Y is —C(CN)— or —N—, in the compound of Formula I or Ia; R₂, R₇, and R₈ are each H, in the compound of Formula I or Ia; R₄ is H or halogen, in the compound of Formula I or Ia; R₃ and R₅ are each halogen, in the compound of Formula I or Ia; or a pharmaceutically acceptable salt thereof.

The present disclosure further provides a method of treating a patient for cancer, comprising administering to a patient in need thereof, an effective amount of a compound of the Formula II.

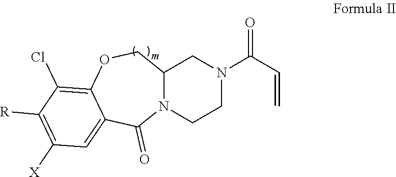

wherein R is

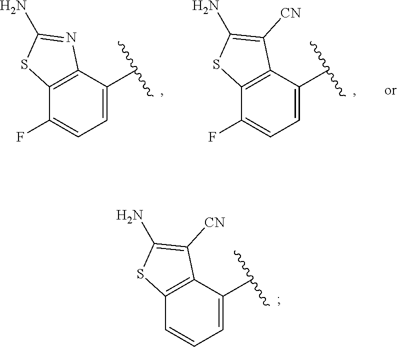 [SMILES: *c1ccc(F)c2sc(N)nc12 ; *c1ccc(F)c2sc(N)c(C#N)c12 ; *c1cccc2sc(N)c(C#N)c12]

X is Cl or F;
and m is 1 or 2;
or a pharmaceutically acceptable salt thereof, and
a SHP2 inhibitor, or a pharmaceutically acceptable salt thereof.

The present disclosure also provides a method of treating a patient for cancer, comprising administering to a patient in need thereof, an effective amount of a compound of the Formula IIa:

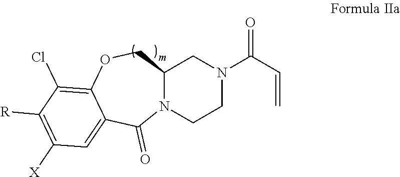

wherein R is

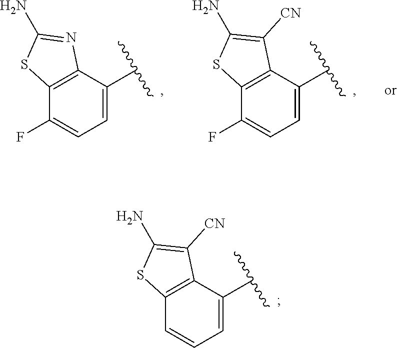 [SMILES: *c1ccc(F)c2sc(N)nc12 ; *c1ccc(F)c2sc(N)c(C#N)c12 ; *c1cccc2sc(N)c(C#N)c12]

X is Cl or F;
and m is 1 or 2;
or a pharmaceutically acceptable salt thereof, and
a SHP2 inhibitor, or a pharmaceutically acceptable salt thereof.

The present disclosure also provides a method of treating a patient for cancer, comprising administering to a patient in need thereof, an effective amount of a compound of Formula I, wherein the compound of Formula I is Formula Ib:

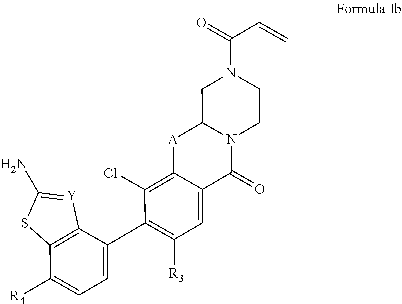

wherein:
   A is —OCH₂— or —OCH₂CH₂—;
   Y is —C(CN)— or —N—;
   R₃ is Cl or F;
   R₄ is H or F when Y is C(CN); and
   R₄ is F when Y is N;
   or a pharmaceutically acceptable salt thereof; and
a SHP2 inhibitor, or a pharmaceutically acceptable salt thereof.

Another way to describe the compound of Formula IIa is with Formula Ib, wherein A is

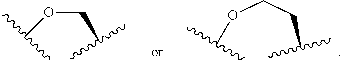

The present disclosure also provides a method of treating a patient for cancer, comprising administering to a patient in need thereof, an effective amount of a compound of Formula I or Ia is selected from any one of Formulae III-VI below:

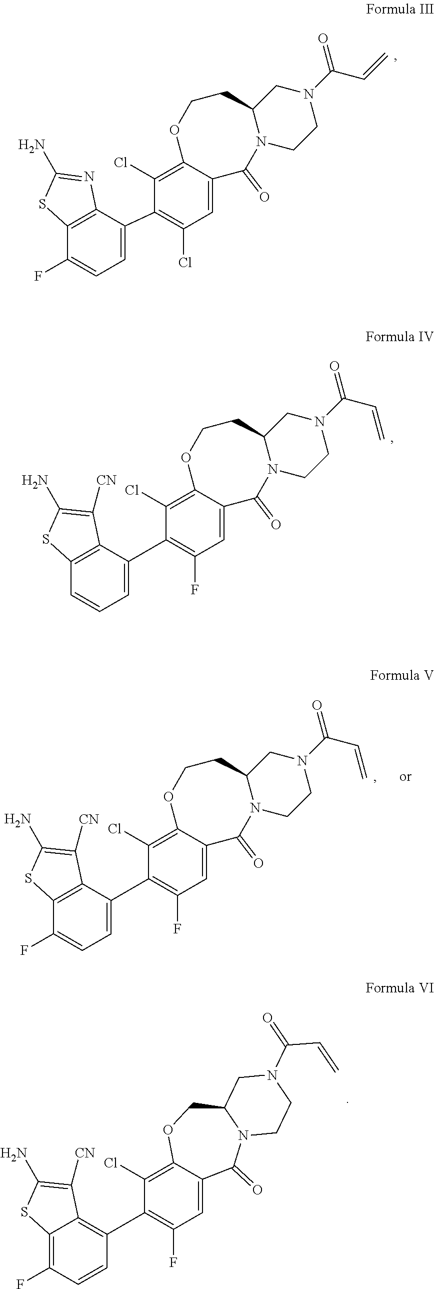 [SMILES: C=CC(=O)N1CCN2C(=O)c3cc(Cl)c(-c4ccc(F)c5sc(N)nc45)c(Cl)c3OCC[C@H]2C1 ; C=CC(=O)N1CCN2C(=O)c3cc(F)c(-c4cccc5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OC[C@H]2C1]

or a pharmaceutically acceptable salt thereof, and
a SHP2 inhibitor, or a pharmaceutically acceptable salt thereof.

In another embodiment, the present disclosure provides a method of treating a patient for cancer, comprising administering to a patient in need thereof, an effective amount of a compound of Formula III which is:

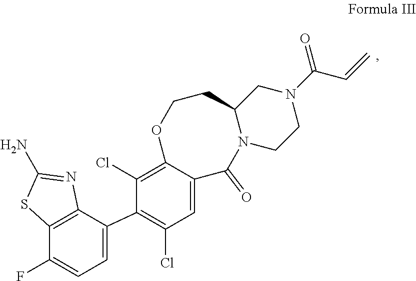 [SMILES: C=CC(=O)N1CCN2C(=O)c3cc(Cl)c(-c4ccc(F)c5sc(N)nc45)c(Cl)c3OCC[C@H]2C1]

or a pharmaceutically acceptable salt thereof, and
a SHP2 inhibitor, or a pharmaceutically acceptable salt thereof.

In another embodiment, the present disclosure provides a method of treating a patient for cancer, comprising administering to a patient in need thereof, an effective amount of a compound of Formula IV which is:

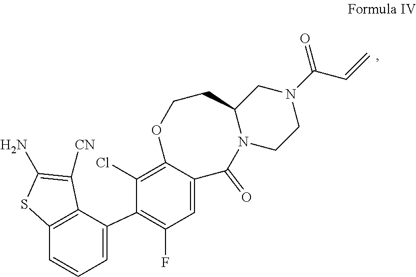 [SMILES: C=CC(=O)N1CCN2C(=O)c3cc(F)c(-c4cccc5sc(N)c(C#N)c45)c(Cl)c3OCC[C@H]2C1]

or a pharmaceutically acceptable salt thereof, and
a SHP2 inhibitor, or a pharmaceutically acceptable salt thereof.

In another embodiment, the present disclosure provides a method of treating a patient for cancer, comprising administering to a patient in need thereof, an effective amount of a compound of Formula V which is:

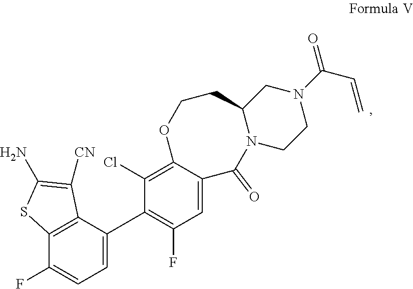 [SMILES: C=CC(=O)N1CCN2C(=O)c3cc(F)c(-c4ccc(F)c5sc(N)c(C#N)c45)c(Cl)c3OCC[C@H]2C1]

or a pharmaceutically acceptable salt thereof, and
a SHP2 inhibitor, or a pharmaceutically acceptable salt thereof.

In another embodiment, the present disclosure provides a method of treating a patient for cancer, comprising administering to a patient in need thereof, an effective amount of a compound of Formula VI which is:

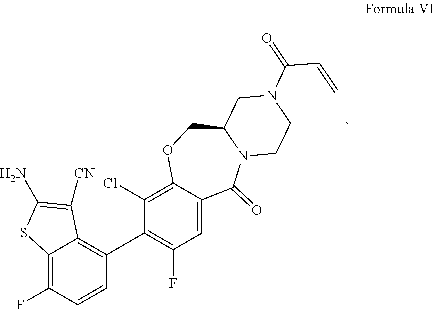 [SMILES: C=CC(=O)N1CCN2C(=O)c3cc(F)c(-c4ccc(F)c5sc(N)c(C#N)c45)c(Cl)c3OC[C@H]2C1]

or a pharmaceutically acceptable salt thereof, and
a SHP2 inhibitor, or a pharmaceutically acceptable salt thereof.

In another embodiment, a method comprising a compound according to any one of Formulae I-VI also includes wherein the SHP2 inhibitor, or a pharmaceutically acceptable salt thereof, is a Type I SHP2 Inhibtor or a Type II SHP2 Inhibitor. In another embodiment, the Type I SHP2 inhibitor is PHPS1 or GS-493, or a pharmaceutically acceptable salt thereof. In another embodiment, the Type I SHP2 inhibitor is NSC-87877 or NSC-117199, or a pharmaceutically acceptable salt thereof. In another embodiment, the Type I SHP2 inhibitor is Cefsulodin, or a pharmaceutically acceptable salt thereof.

In another embodiment, the Type II SHP2 inhibitor is JAB-3068 or JAB-3312, or a pharmaceutically acceptable salt thereof. In another embodiment, the Type II SHP2 inhibitor is RMC-4550 or RMC-4630, or a pharmaceutically acceptable salt thereof. In another embodiment, the Type II SHP2 inhibitor is a SHP099, SHP244, SHP389, SHP394, or TN0155, or a pharmaceutically acceptable salt thereof. In another embodiment, the Type II SHP2 inhibitor is RG-6433 or RLY-1971, or a pharmaceutically acceptable salt thereof.

In another embodiment, the SHP2 inhibitor is BBP-398, IACS-15509, or IACS-13909, or a pharmaceutically acceptable salt thereof. In another embodiment, the SHP2 inhibitor is X37, or a pharmaceutically acceptable salt thereof. In another embodiment, the SHP2 inhibitor is ERAS-601, or a pharmaceutically acceptable salt thereof. In another embodiment, the SHP2 inhibitor is SH3809, or a pharmaceutically acceptable salt thereof. In another embodiment, the SHP2 inhibitor is HBI-2376, or a pharmaceutically acceptable salt thereof. In another embodiment, the SHP2 inhibitor is ETS-001, or a pharmaceutically acceptable salt thereof. In another embodiment, the SHP2 inhibitor is PCC0208023, or a pharmaceutically acceptable salt thereof.

In another embodiment the present disclosure provides a method of treating a patient for cancer, comprising administering to a patient in need thereof, an effective amount of a compound according to any one of Formulae I-VI, or a pharmaceutically acceptable salt thereof, with RMC-4630.

In another embodiment the present disclosure provides a method of treating a patient for cancer, comprising administering to a patient in need thereof, an effective amount of a compound according to any one of Formulae I-VI, or a pharmaceutically acceptable salt thereof, with JAB-3068.

In another embodiment the present disclosure provides a method of treating a patient for cancer, comprising administering to a patient in need thereof, an effective amount of a compound according to any one of Formulae I-VI, or a pharmaceutically acceptable salt thereof, with TN0155.

The present disclosure also provides a method of treating a patient for cancer, comprising administering to a patient in need thereof, an effective amount of a compound according to any one of Formulae I-VI, or a pharmaceutically acceptable salt thereof, with a SHP2 inhibitor, or a pharmaceutically acceptable salt thereof.

In various embodiments, the cancer is lung cancer, colorectal cancer, pancreatic cancer, bladder cancer, cervical cancer, endometrial cancer, ovarian cancer, cholangiocarcinoma, or esophageal cancer. In preferred embodiments, the cancer is non-small cell lung cancer, pancreatic cancer, or colorectal cancer. In still more preferred embodiments, the cancer is non-small cell lung cancer.

The present disclosure also provides a method of treating a patient with a cancer comprising administering to a patient in need thereof an effective amount of a compound according to any one of Formulae I-VI, or a pharmaceutically acceptable salt thereof, with a SHP2 inhibitor, or a pharmaceutically acceptable salt thereof, in which the cancer has one or more cells that express a mutant KRas G12C protein with or without a SHP2 dysregulation or overexpression. The present disclosure also provides a method of treating cancer, comprising administering to a patient in need thereof, an effective amount of a compound according to any one of Formulae I-VI, or a pharmaceutically acceptable salt thereof, and a SHP2 inhibitor compound, or a pharmaceutically acceptable salt thereof, wherein the cancer is non-small cell lung cancer, and wherein one or more cells with or without a SHP2 dysregulation or overexpression express KRas G12C mutant protein. The present disclosure also provides a method of treating cancer, comprising administering to a patient in need thereof, an effective amount of a compound according to any one of Formulae I-VI, or a pharmaceutically acceptable salt thereof, and a SHP2 inhibitor compound, or a pharmaceutically acceptable salt thereof, wherein the cancer is colorectal cancer, and wherein one or more cells with or without a SHP2 dysregulation or overexpression express KRas G12C mutant protein. The present disclosure also provides a method of treating cancer, comprising administering to a patient in need thereof, an effective amount of a compound according to any one of Formulae I-VI, or a pharmaceutically acceptable salt thereof, and a SHP2inhibitor compound, or a pharmaceutically acceptable salt thereof, wherein the cancer is pancreatic cancer, and wherein one or more cells with or without a SHP2 dysregulation or overexpression express KRas G12C mutant protein.

The present disclosure also provides a method of treating cancer in a patient in need thereof, wherein the patient has a cancer that was determined to express the KRas G12C mutant protein and a SHP2 dysregulation or overexpression.

In another embodiment, the cancer is non-small cell lung carcinoma, in which the cancer has one or more cells that express a KRas G12C mutant protein and/or a SHP2 dysregulation or overexpression. In another embodiment, the cancer is colorectal carcinoma in which the cancer has one or more cells that express a KRas G12C mutant protein and/or a SHP2 dysregulation or overexpression. In yet another embodiment, the cancer is mutant pancreatic cancer in which the cancer has one or more cells that express a KRas G12C mutant protein and/or a SHP2 dysregulation or overexpression. In another embodiment, the present disclosure comprising a method of treating KRas G12C mutant bearing cancers of other origins and/or a SHP2 dysregulation or overexpression.

In still yet another embodiment, the present disclosure comprises a method of treating cancer, comprising administering to a patient in need thereof, an effective amount of a compound according to any one of Formulae I-VI, or a pharmaceutically acceptable salt thereof, with a SHP2 inhibitor, or a pharmaceutically acceptable salt thereof, in which the cancer has one or more cells that express a mutant KRas G12C protein or a SHP2 dysregulation or overexpression. In some embodiments, the patient has a cancer that was determined to have one or more cells expressing the KRas G12C mutant protein prior to administration of the compound, or a pharmaceutically acceptable salt thereof, or the SHP2 inhibitor, or a pharmaceutically acceptable salt thereof. In some embodiments, the patient has a cancer that has a KRAS G12C mutation.

In still yet another embodiment, the present disclosure comprises a method of treating cancer, comprising administering to a patient in need thereof, an effective amount of a compound according to any one of Formulae I-VI, or a pharmaceutically acceptable salt thereof, with a SHP2 inhibitor, or a pharmaceutically acceptable salt thereof, wherein the compound of the formula and the SHP2 inhibitor are provided in simultaneous or sequential combination to the patient in need thereof. In some embodiments, the compound of the formula and the SHP2 inhibitor are provided in simultaneous combination to the patient in need thereof. In some embodiments, the compound of the formula and the SHP2 inhibitor are provided in sequential combination to the patient in need thereof. In some embodiments, the compound of the formula is provided to the patient in need thereof before the SHP2 inhibitor is provided to the patient in need thereof. In some embodiments, the SHP2 inhibitor is provided to the patient in need thereof before the compound of the formula is provided to the patient in need thereof.

In the methods described herein, the cancer can be lung cancer, colorectal cancer, pancreatic cancer, bladder cancer, cervical cancer, endometrial cancer, ovarian cancer, cholangiocarcinoma, or esophageal cancer. In preferred embodiments, the cancer is non-small cell lung cancer, pancreatic cancer, or colorectal cancer. In still more preferred embodiments, the cancer is non-small cell lung cancer. In other embodiments, the cancer has one or more cancer cells that express the mutant KRas G12C protein and/or a SHP2 dysregulation or overexpression. Preferably, the cancer is selected from KRas G12C mutant non-small cell lung cancer, KRas G12C mutant colorectal cancer, and KRas G12C mutant pancreatic cancer.

In an embodiment the present disclosure provides a compound according to any one of Formulae I-VI, or a pharmaceutically acceptable salt thereof, for use in therapy in simultaneous, separate, or sequential combination with a SHP2 inhibitor, or a pharmaceutically acceptable salt thereof. The present disclosure also provides a compound according to any one of Formulae I-VI, or a pharmaceutically acceptable salt thereof, for use in the treatment of cancer in simultaneous, separate, or sequential combination with a SHP2 inhibitor, or a pharmaceutically acceptable salt thereof. The present disclosure also provides for the use of a compound according to any one of Formulae I-VI, or a pharmaceutically acceptable salt thereof, in the manufacture of a medicament for the treatment of cancer in simultaneous, separate, or sequential combination with a SHP2 inhibitor, or a pharmaceutically acceptable salt thereof, in the manufacture of a medicament for the treatment of cancer.

The term “KRas G12Ci” as used herein may refer to any compound according to any one of Formulae I-VI, or a pharmaceutically acceptable salt thereof.

The term “pharmaceutically acceptable salt” as used herein refers to a salt of a compound considered to be acceptable for clinical and/or veterinary use. Examples of pharmaceutically acceptable salts and common methodology for preparing them can be found in “Handbook of Pharmaceutical Salts: Properties, Selection and Use” P. Stahl, et al., 2nd Revised Edition, Wiley-VCH, 2011 and S. M. Berge, et al., “Pharmaceutical Salts”, Journal of Pharmaceutical Sciences, 1977, 66(1), 1-19.

The pharmaceutical compositions for the present disclosure may be prepared using pharmaceutically acceptable additives. The term “pharmaceutically acceptable additive(s)” as used herein for the pharmaceutical compositions, refers to one or more carriers, diluents, and excipients that are compatible with the other additives of the composition or formulation and not deleterious to the patient. Examples of pharmaceutical compositions and processes for their preparation can be found in “Remington: The Science and Practice of Pharmacy”, Loyd, V., et al. Eds., 22^{nd} Ed., Mack Publishing Co., 2012. Non-limiting examples of pharmaceutically acceptable carriers, diluents, and excipients include the following: saline, water, starch, sugars, mannitol, and silica derivatives; binding agents such as carboxymethyl cellulose, alginates, gelatin, and polyvinyl-pyrrolidone; kaolin and bentonite; and polyethyl glycols.

As used herein, the term “effective amount” refers to an amount that is a dosage, which is effective in treating a disorder or disease, such as a cancerous lesion or progression of abnormal cell growth and/or cell division. The attending physician, as one skilled in the art, can readily determine an effective amount by the use of conventional techniques and by observing results obtained under analogous circumstances.

Dosages per day of treatment for the compound of Formula I normally fall within a range of between about 1 mg per day or twice daily and 1000 mg per day or twice daily, more preferably 100 mg per day or twice daily and 900 mg per day or twice daily.

Dosages per day of treatment for the SHP2 inhibitor, JAB-3068 or JAB-3312, RMC-4550 or RMC-4630, SHP099 or TN0155, RG-6433 or RLY-1971, BBP-398, IACS-15509, or IACS-13909, X37, ERAS-601, SH3809, HBI-2376, or ETS-001. normally fall within the range of about 0.1 to about 100 mg. In some instances, dosage levels below the lower limit of this range may be more than adequate, while in other cases still larger doses may be employed for the SHP2 inhibitor, JAB-3068 or JAB-3312, RMC-4550 or RMC-4630, SHP099 or TN0155, RG-6433 or RLY-1971, BBP-398, IACS-15509, or IACS-13909, X37, ERAS-601, SH3809, HBI-2376, or ETS-001. Preferred dosages fall within the range of 1 to 80 mg; more preferably between 1 and 50 mg; still more preferably between 1 and 30 mg; still yet more preferably between 1 to 25 mg, for the SHP2 inhibitor, JAB-3068 or JAB-3312, RMC-4550 or RMC-4630, SHP099 or TN0155, RG-6433 or RLY-1971, BBP-398, IACS-15509, or IACS-13909, X37, ERAS-601, SH3809, HBI-2376, or ETS-001. The dosages can be administered once, twice, three times or more daily. In one embodiment, TN0155, can be administered at a dosage of 20 mg per dose administered orally twice a day (BID). In one embodiment, the dosage regiment for TNO155 includes two weeks of daily administration followed by one week without administration.

Factors considered in the determination of an effective amount or dose of a compound include: whether the compound or its salt will be administered; the co-administration of other agents, if used; the species of patient to be treated; the patient's size, age, and general health; the degree of involvement or stage and/or the severity of the disorder; the response of the individual patient; the mode of administration; the bioavailability characteristics of the preparation administered; the dose regimen selected; and the use of other concomitant medication.

A treating physician, veterinarian, or other medical person will be able to determine an effective amount of the compound for treatment of a patient in need. Preferred pharmaceutical compositions can be formulated as a tablet or capsule for oral administration, a solution for oral administration, or an injectable solution. The tablet, capsule, or solution can include a compound of the present disclosure in an amount effective for treating a patient in need of treatment for cancer.

As used herein, the terms “treating”, “to treat”, or “treatment”, includes slowing, reducing, or reversing the progression or severity of an existing symptom, disorder, condition, which can include specifically slowing the growth of a cancerous lesion or progression of abnormal cell growth and/or cell division.

As used herein, the term “patient” refers to a mammal in need of treatment. Preferably, the patient is a human that is in need of treatment for cancer, for example, KRas G12C mutant bearing cancers.

Individual isomers, enantiomers, diastereomers, and atropisomers may be separated or resolved at any convenient point in the synthesis of compounds listed below, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen,” Stereochemistry of Organic Compounds”, Wiley-Interscience, 1994). The present disclosure includes certain compounds, which are atropisomers and which can exist in different conformations or as different rotomers. Atropisomers are compounds, which exist in different conformations arising from restricted rotation about a single bond. Atropisomers can be isolated as separate chemical species if the energy barrier to rotation about the single is sufficiently high enough and the rate of interconversion is slow enough to allow the individual rotomers to be separated from each other. The present disclosures contemplates all of the isomers, enantiomers, diastereomers, and atropisomers disclosed herein or that could be made using the compounds disclosed herein.

Any compound according to any one of Formulae I-VI is readily converted to and may be isolated as a pharmaceutically acceptable salt. Salt formation can occur upon the addition of a pharmaceutically acceptable acid to form the acid addition salt. Salts can also form simultaneously upon deprotection of a nitrogen or oxygen, i.e., removing the protecting group. Examples, reactions and conditions for salt formation can be found in Gould, P. L., “Salt selection for basic drugs,” International Journal of Pharmaceutics, 33: 201-217 (1986); Bastin, R. J., et al. “Salt Selection and Optimization Procedures for Pharmaceutical New Chemical Entities,” Organic Process Research and Development, 4: 427-435 (2000); and Berge, S. M., et al., “Pharmaceutical Salts,” Journal of Pharmaceutical Sciences, 66: 1-19, (1977).

The compounds of the present disclosure, or salts thereof, may be prepared by a variety of procedures, some of which are illustrated in the Preparations and Examples below. The specific synthetic steps for each of the routes described may be combined in different ways, or in conjunction with steps from different routes, to prepare compounds or salts of the present disclosure. The products of each step in the Preparations below can be recovered by conventional methods, including extraction, evaporation, precipitation, chromatography, filtration, trituration, and crystallization.

### Biological Assays

The following assays demonstrate that the combination of exemplified compounds described herein are inhibitors of KRas G12C and inhibit growth of certain tumors in vitro and/or in vivo.

The statistics methods used for the in vivo models are listed below.

### Tumor Growth Inhibition Analysis

Tumor volumes were transformed to a log₁₀ scale to equalize variance across time and treatment. Log₁₀ volume and body weight were separately analyzed using a two-way repeated measures analysis of variance model (RM ANOVA) consisting of time, treatment, and the interaction between time and treatment using the MIXED procedure of the SAS software package (Version 9.3). Spatial Power covariance structure was used to model the correlation of observations across time for the same subject. Kenward-Roger (1997) denominator degrees of freedom (DDFM) calculations were used for tests of fixed effects. Post-hoc pairwise t-tests were used to compare tumor volumes and body weights of treated groups to the control group on the summarized day, p-values≤0.05 were considered statistically significant. The MIXED procedure was also used separately for each treatment group to calculate least squares means (LS Means) and standard errors for each time point for the purpose of plotting and inclusion in summary tables.

### Efficacy Calculations

Efficacy was calculated at the end of the treatment if at least half of the initial number of subjects in the control group remained in the study. Otherwise, efficacy was calculated on the most recent observation day prior to the end of treatment where these conditions were met.

### % Delta T/C

% Delta[TIC] was defined as 100 times the ratio of the tumor volume change from Baseline at time t of the treated group versus the tumor volume change from Baseline of the control group at time t, where t is greater than t_{Baseline} and treated group change from Baseline is greater than zero (equation 1)

$$\% Delta [ T / C ] = 100 \times \frac{Δ {T}_{t}}{Δ {C}_{t}} \qquad ( 1 )$$

where,
   TV_{t,x} is the tumor volume of group X at time t, t>t_{Baseline}
   ΔTₜ=TV_{t,Trmnt}−TV_{Baseline}, ΔTₜ>0
   ΔCₜ=TV_{t,Ctrl}−TV_{Baseline}, ΔCₜ>0
   TV_{Baseline} is the grand mean of all tumors at t_{Baseline}.

### % Tumor Regression

% Regression was defined as 100 times the ratio of the tumor volume change from Baseline of the treated group versus Baseline tumor volume at time t, where t is greater than t_{Baseline} and treated group change from Baseline is less than or equal to zero (equation 2)

$$\% Regression = 100 \times \frac{Δ {T}_{t}}{{TV}_{Baseline}} , t > {t}_{Baseline} \qquad ( 2 )$$

### % Tumor Growth Inhibition (TGI)

% TGI was defined as 100 minus % Delta TIC or % Regression as applicable (equation 3)

$$\% TGI = \{ \begin{matrix}100 - \% Delta [ T / C ] , & Δ {T}_{t} > 0 \\ 100 - \% Regression , & Δ {T}_{t} \leq 0\end{matrix} \qquad ( 3 )$$

### Combination Analysis

The Bliss Independence method was used to estimate the combination treatment effect on tumor volume. Log₁₀ tumor volume data for the control group, combination groups, and their respective single agent treatment groups were analyzed using RM ANOVA, as previously described, but with an added two-way interaction effect contrast estimate of the following form:

TV₀−TV₁−TV₂+TV_{1,2}
where, TV₀, TV₁, TV₂, and TV_{1,2} are the estimated log₁₀ volume LSMeans for the control group (0), each single agent alone treated group (1 or 2), and the combination treated group (1,2).

If the combination effect is exactly additive then the contrast estimate equals 0. This approach is mathematically equivalent to the Bliss Independence method assuming that tumors can, in theory, reach zero (complete regression).

If the contrast estimate is significantly different from zero (p≤0.05), then the combination effect is greater than (synergistic) or less than (antagonistic) additive if the observed combination group mean volume is less than or greater than the expected additive response (EAR) volume.

$$EAR = \frac{{TV}_{1} \times {TV}_{2}}{{TV}_{0}}$$

If the interaction contrast test and all pairwise comparisons versus the combination group are all statistically significant (p≤0.05), and the observed combination mean volume is less than the EAR volume, then the combination effect is declared greater than additive (synergistic).

If the interaction contrast test is statistically significant (p≤0.05) and the observed combination mean volume is greater than the EAR volume, then the combination effect is declared less than additive (antagonistic) regardless of the pairwise comparison results.

If the interaction test is not statistically significant (p≥0.05), but all pairwise comparisons versus the combination group are significant (p≤0.05), then the combination effect is declared additive, otherwise the combination effect is inconclusive (no effect).

Combination Studies with KRas G12Ci and RMC-4550

The purpose of this study was to evaluate the anti-tumor efficacy of KRAS G12C mutant-selective inhibitor KRas G12C₁, in combination with SHP2 inhibitor RMC-4550, in human NSCLC tumor or PDX models, and a human CRC xenograft model, each of which harbors a KRAS G12C mutation.

The combination of KRAS G12CI with another SHP2 inhibitor (RMC-4550) showed robust synergy in multiple NSCLC xenograft and PDX models.

A study evaluated the combination efficacy of KRas G12Ci with SHP2 inhibitor RMC-4550. The combination efficacy was evaluated in two lung cancer xenograft models (H358 and H1373), one lung cancer PDX model (EL3187), one colorectal xenograft models (SW837). The combination of KRas G12Ci and SHP2 inhibitor showed synergy and significantly better anti-tumor efficacy than either monotherapy. In three lung cancer models (EL3187, H358 and H1373) and colorectal cancer SW837 model, better anti-tumor activity and significant tumor regression was observed by combinational therapy.

### Test Articles:

KRas G12Ci: 10% NMP, 90% of 15% w/v PVP-VA in PEG400, was administered by oral gavage (0.2 mL/animal). 10% of the total vehicle volume of NMP was added to pre weighed test article. Mixing was done until all test article dissolved (no visible particles).
QS with the PVPVA/PEG400 vehicle and mix. Batch Weekly.
SHP2 (RMC-4550): 20% Captisol (w/v) in 25 mM Phosphate Buffer, pH 2.0 to 2.2, was administered by oral gavage (0.2 mL/animal). A portion of the vehicle (approximately 20%) was added to the test article and was mixed to wet. The remainder of the vehicle, minus a small volume, was added and mixed. Probe sonicated on an ice bath to reduce particle size. QS to final volume and mix. Batch Weekly.
Cell lines used: H358, H1373 and SW837 cells.

TABLE 1
Experimental Design

| | | | | Xenograft Model | | | |
|---|---|---|---|---|---|---|---|
| | | Dose | Dose | Study 1 | Study 2 | Study 3 | Study 4 |
| Groups | Treatment | (mg/kg) | Schedule | (n = 5) | (n = 5) | (n = 5) | (n = 5) |
| Group 1 | Vehicle | N/A | PO, QDx28 | H358^{e} | H1373^{b} | EL3187^{d} | SW837^{e} |
| Group 2 | KRas G12Ci | 3 | PO, QDx28 | N/A | N/A | EL3187 | N/A |
| | | 10 | PO, QDx28 | H358 | H1373 | N/A | SW837 |
| Group 3 | RMC-4550 | 30 | PO, QDx28 | H358 | H1373^{c} | EL3187 | SW837 |
| Group 4a | KRas G12Ci + | 3 + 30 | PO, QDx28 | N/A | N/A | EL3187 | N/A |
| | RMC-4550 | | | | | | |
| Group 4b | KRas G12Ci + | 10 + 30 | PO, QDx28 | H358^{a} | H1373 | N/A | SW837 |
| | RMC-4550 | | | | | | |

^{a}In the H358 combination treatment group, one animal was reported to have been sacrificed due to moribund after 3 days of dosing.
^{b}In the H1373 vehicle group, two animals were sacrificed on Day 17 of treatment and one animal was sacrificed on Day 24 of treatment due to tumor burden; the rest of the vehicle treated group was sacrified on Day 24.
^{c}In the RMC-4550 treatment group, one animal was sacrificed on Day 21 due to tumor burden.
^{d}In the EL3187 vehicle group, three animals were sacrificed on Day 22 of treatment due to tumor burden.
^{e}In the H358 and SW837 studies, the vehicle group n = 6.
N/A, not applicable; n, number of animals per group.

### Mouse Xenograft Models

Female athymic nude mice (Envigo RMS, Inc., Mount Comfort, Indiana), or NOD SCID gamma mice (The Jackson Laboratory, Bar Harbor, Maine), weighing 20 to 22 grams, were used for the studies. The animals were housed and were provided free access to standard diet and water. For H358, H1373, and SW837 xenograft tumor growth, 5×106 cells in a volume of 0.2 mL Hanks' Balanced Salt solution (HBSS):Matrigel (Corning, Cat #354234) (1:1) were implanted subcutaneously in the right flank of each animal. H358 cells were implanted into NOD SCID gamma mice and H1373 and SW837 cells were implanted into athymic nude mice. Tumor volumes were measured using calipers twice weekly. When the tumor volumes reached 200-300 mm³ the mice were randomized (n=5 per group) based on tumor measurement and body weight using the multi-task block randomization tool. Treatment was initiated with oral administration (gavage) of either 0.2 mL vehicle, KRas G12Ci at 10 mg/kg QD, RMC-4550 at 30 mg/kg QD, or the combination of 0.2 mL KRas G12Ci at 10 mg/kg QD and 0.2 mL RMC-4550 at 30 mg/kg QD for 28 days, according to the experimental design shown in Table 1. In the H358 combination treatment group, one animals was sacrificed due to moribound after 6 days of dosing. In the H1373 model, two animals in the vehicle group were sacrificed at Day 17 and one at Day 24 of treatment due to tumor necrosis or tumor burden, respectively; one animal in the RMC-4550 group was sacrificed on Day 21 due to tumor necrosis. Statistical analysis results were summarized at Day 28 of treatment for the H358 and SW837 xenograft studies, and at Day 24 of treatment for the H1373 xenograft study. Tumor regrowth was monitored for an additional 10-18 days post last dose in the H358 and H1373 studies.

### EL3187 PDX Model

EL3187 tumor fragments were obtained from the Methodist Research Institute Biorepository. Frozen vials containing the tumor fragments were thawed at 37° C. in a water bath. The tumor fragments were transferred to a 50 mL Falcon tube and then ice-cold DMEM medium was slowly added to a total volume of 35 mL. The tumor fragments were then centrifuged at 130×g for 2 minutes at 4° C. and the supernatant was aspirated. This washing step was repeated twice and the tumor fragments were resuspended in 10 mL DMEM. The tumor fragments were subcutaneously implantated into the right rear flank of female 6-8 week old (20-22 gram) athymic nude-Foxnlnu feeder mice (from Envigo RMS, Inc., Mount Comfort, Indiana). Once tumor volumes reached 800-1000 mm3, the animals were sacrificed and the tumors were harvested using aseptic technique. Fresh tumors, passage 4, were cut into 10-15 mm3 fragments and placed into cold Gibco Hibernate Medium, and then the pooled tumor fragments were subcutaneously implanted into animals with a 10 g trochar needle. On Day 24 post-implantation, when the tumor size was approximately 250-350 mm3, the mice were randomized (n=5 per group) according to the experimental design shown in Table 1. Groups were dosed by oral gavage with 0.2 mL of either vehicle, KRas G12Ci at 3 mg/kg QD, RMC-4550 at 30 mg/kg QD, or the combination of 0.2 mL KRas G12Ci at 3 mg/kg QD and 0.2 mL RMC-4550 at 30 mg/kg QD for 28 days. Tumor volumes were measured using calipers twice weekly. Three out of five animals in the vehicle group were sacrificed on Day 46 post-implant (Day 22 of treatment) due to tumor burden. Statistical analysis results for the EL3187 PDX study were summarized at Day 22 of treatment. Tumor regrowth was monitored for an additional 30 days post last dose.

TABLE 2
Tumor Growth Inhibition Activity of KRas G12Ci and SHP2 inhibitor
RMC-4550 Combination Treatment in the H358 NSCLC Xenograft Model

| | | | | Tumor Volume | | | | | | |
|---|---|---|---|---|---|---|---|---|---|---|
| | | | | | | | TGI (%) or | Body Weight | | |
| | | Dose | | Mean | | | Regression | Mean | | |
| Treatment | mg/kg | Frequency | n | (mm³) | SE | p-value | (−%) | (g) | SE | p-value |
| Vehicle | N/A | PO, QDx28 | 6 | 1366.4 | 163.1 | N/A | N/A | 22.6 | 1.1 | N/A |
| KRas G12Ci | 10 | PO, QDx28 | 5 | 539.4 | 80.8 | <.0001 | 74.9 | 24.8 | 0.7 | 0.095 |
| RMC-4550 | 30 | PO, QDx28 | 5 | 574.6 | 95.6 | <.0001 | 71.7 | 26.8 | 0.7 | 0.001 |
| KRas G12Ci + | 10 + 30 | PO, QDx28 | 4 | 91.5 | 8.8 | <.0001 | −65.1 | 25.1 | 1.0 | 0.070 |
| RMC-4550 | | | | | | | | | | |

P-values were estimated from RM ANOVA post-hoc pairwise t-tests between vehicle and each treatment group. Statistical analysis results were summarized on treatment Day 28, p ≤ 0.05 was considered statistically significant.
n = number of animals per group included in the Day 28 statistical analysis; N/A, not applicable; PO, by mouth; QD, once daily; TGI, tumor growth inhibition; SE, standard error.

TABLE 3
Tumor Growth Inhibition Activity of KRas G12Ci and RMC-4550
Combination Treatment in the H1373 NSCLC Xenograft Model

| | | | | Tumor Volume (day 24 of treatment) | | | | | Body Weight | | |
|---|---|---|---|---|---|---|---|---|---|---|---|
| | | Dose | | Mean | | | TGI | Regression | Mean | | |
| Treatment | mg/kg | Frequency | n | (mm³) | SE | p-value | (%) | (−%) | (g) | SE | p-value |
| Vehicle | N/A | PO, QDx28 | 3 | 1368.3 | 289.0 | N/A | N/A | N/A | 26.9 | 0.7 | N/A |
| KRas G12Ci | 10 | PO, QDx28 | 5 | 239.6 | 34.4 | <.0001 | 98.5 | N/A | 26.9 | 0.8 | 0.9562 |
| RMC-4550 | 30 | PO, QDx28 | 4 | 449.7 | 74.6 | 0.0001 | 80.2 | N/A | 27.4 | 0.5 | 0.6427 |
| KRas G12Ci + | 10 + 30 | PO, QDx28 | 5 | 57.9 | 8.0 | <.0001 | N/A | −74 | 26.6 | 0.4 | 0.8322 |
| RMC-4550 | | | | | | | | | | | |

P-values were estimated from RM ANOVA post-hoc pairwise t-tests between vehicle and each treatment group. Statistical analysis results were summarized on treatment Day 24, p ≤ 0.05 was considered statistically significant.
n = number of animals per group included in the Day 24 statistical analysis; N/A, not applicable; PO, by mouth; QD, once daily; TGI, tumor growth inhibition; SE, standard error.

TABLE 4
Tumor Growth Inhibition Activity of KRas G12Ci and RMC-4550
Combination Treatment in the SW837 CRC Xenograft Model

| | | | | Tumor Volume | | | | | Body Weight | | |
|---|---|---|---|---|---|---|---|---|---|---|---|
| | | Dose | | Mean | | | TGI | Regression | Mean | | |
| Treatment | mg/kg | Frequency | n | (mm³) | SE | p-value | (%) | (−%) | (g) | SE | p-value |
| Vehicle | N/A | PO, QDx28 | 6 | 526.6 | 67.6 | N/A | N/A | N/A | 26.7 | 0.8 | N/A |
| KRas G12Ci | 10 | PO, QDx28 | 5 | 325.9 | 31.6 | 0.006 | 70.6 | N/A | 26.9 | 0.8 | 0.8689 |
| RMC-4550 | 30 | PO, QDx28 | 5 | 300.8 | 25.3 | 0.0015 | 79.4 | N/A | 28.7 | 0.8 | 0.0515 |
| KRas G12Ci + | 10 + 30 | PO, QDx28 | 5 | 159.8 | 18.5 | <.0001 | N/A | −34 | 27.3 | 0.4 | 0.5617 |
| RMC-4550 | | | | | | | | | | | |

P-values were estimated from RM ANOVA post-hoc pairwise t-tests between vehicle and each treatment group. Statistical analysis results were summarized on treatment Day 28, p ≤ 0.05 was considered statistically significant.
n = number of animals per group included in the Day 28 statistical analysis; N/A, not applicable; PO, by mouth; QD, once daily; TGI, tumor growth inhibition; SE, standard error.

TABLE 5
Tumor Growth Inhibition Activity of KRas G12Ci and RMC-
4550 Combination Treatment in the EL3187 NSCLC PDX Model

| | | | | Tumor Volume (day 22 of treatment) | | | | | Body Weight | | |
|---|---|---|---|---|---|---|---|---|---|---|---|
| | | Dose | | Mean | | | TGI | Regression | Mean | | |
| Treatment | mg/kg | Frequency | n | (mm³) | SE | p-value | (%) | (−%) | (g) | SE | p-value |
| Vehicle | N/A | PO, QDx28 | 5 | 2062.0 | 267.3 | N/A | N/A | N/A | 28.1 | 0.8 | N/A |
| KRas G12Ci | 10 | PO, QDx28 | 5 | 524.2 | 179.2 | 0.0173 | 84.8 | N/A | 26.7 | 0.8 | 0.2288 |
| RMC-4550(SHP2) | 30 | PO, QDx28 | 5 | 731.6 | 150.2 | 0.0284 | 73.4 | N/A | 27.7 | 0.9 | 0.7694 |
| KRas G12Ci + | 3 + 30 | PO, QDx28 | 5 | 59.3 | 39.6 | <.0001 | N/A | −76.2 | 27.8 | 1.2 | 0.7826 |
| RMC-4550 | | | | | | | | | | | |

P-values were estimated from RM ANOVA post-hoc pairwise t-tests between vehicle and each treatment group. Statistical analysis results were summarized on treatment Day 22, p ≤ 0.05 was considered statistically significant.
n = number of animals per group included in the Day 22 statistical analysis; N/A, not applicable; PO, by mouth; QD, once daily; TGI, tumor growth inhibition; SE, standard error.

### Results and Discussion

In this study, the anti-tumor activity of the KRAS G12C inhibitor in combination with the SHP2 inhibitor RMC-4550 was evaluated in two NSCLC models (H358 and H1373), one NSCLC patient-derived xenograft (PDX) model (EL3187), and one CRC xenograft model (SW837). In the H358, H1373 and SW837 xenograft models, tumor-bearing mice were treated with either KRas G12Ci at 10 mg/kg once daily (QD), RMC-4550 at 30 mg/kg QD, or the combination of KRas G12Ci at 10 mg/kg QD and RMC-4550 at 30 mg/kg QD (Table 1). KRas G12Ci at 10 mg/kg QD was selected as a sub-optimal dose based on preclinical efficacy studies when used as a monotherapy in these models, while RMC-4550 at 30 mg/kg QD was chosen according to publication in preclinical xenograft models.

In the H358 NSCLC xenograft model, treatment with either KRas G12Ci or RMC-4550 for 28 days resulted in significant tumor growth inhibition (TGI) of 74.9% or 71.7%, respectively, whereas treatment with KRas G12Ci in combination with RMC-4550 for the same period of time resulted in significant tumor regression (TR) of 65.1% (Table 2). The anti-tumor activity of the dual treatment combination was significantly greater than that of either agent alone, and the Expected additive response (EAR), indicating that the combination treatment effect was synergistic in this model. After treatment withdrawal in the combination treatment group, sustained tumor regression was maintained for approximately 10 days. Although no animal weight loss was observed in any of the treatment groups and there was no significant difference in mean body weight between any of the compound treatment groups and the vehicle group on treatment Day 28 (Table 2). In the combination treatment group, one animal was sacrificed due to moribound after 6 days of dosing.

In the H1373 NSCLC model, two animals in the vehicle group were sacrificed at Day 17 and one at Day 24 of treatment due to tumor necrosis or tumor burden, respectively; one animal in the RMC-4550 group was also sacrificed on Day 21 due to tumor necrosis. Data at Day 24 of treatment was chosen for statistical analysis. In this model, treatment with either KRas G12Ci or RMC-4550 for 24 days resulted in significant TGI of 98.5% or 80.2%, respectively, whereas the combination treatment resulted in significant TR of 74% (Table 3). While the anti-tumor activity of KRas G12Ci in combination with RMC-4550 was significantly greater than that of either agent alone, results of the combination interaction test indicated an additive combination treatment effect. Tumor regression was sustained for about 10 days in the combination treatment group after withdrawal of compound treatment. No animal weight loss was observed in any of the treatment groups and there was no significant difference in mean body weight between any of the compound treatment groups and the vehicle group on treatment Day 24 (Table 3).

In the SW837 CRC model, treatment with either KRas G12Ci or RMC-4550 alone for 28 days resulted in significant TGI of 70.6% or 79.4%, respectively, whereas the combination treatment resulted in significant TR of 34% (Table 4). Similar to the results above, the anti-tumor activity of the combination treatment was significantly greater than that of either agent alone, and the combination treatment effect was shown to be additive. No animal weight loss was observed in any of the treatment groups in this model and there was no significant difference in mean body weight between the compound and vehicle treatment groups on treatment Day 28 (Table 4).

Using the EL3187 NSCLC PDX model, tumor-bearing mice were treated with either KRas G12Ci at 3 mg/kg QD, RMC-4550 at 30 mg/kg QD, or the combination of KRas G12Ci and RMC-4550 at these dosages (Table 1). KRas G12Ci at 3 mg/kg QD was selected as the sub-optimal dose based on previous preclinical efficacy studies when used as a monotherapy in this model. In this study, three out of five animals in the vehicle group were sacrificed on Day 46 post implant (Day 22 of treatment) due to tumor burden. Data at Day 22 of treatment was chosen for statistical analysis. Treatment with either KRas G12Ci or RMC-4550 alone for 22 days resulted in significant TGI of 84.8% or 73.4%, respectively, whereas the combination treatment resulted in significant TR of 76.2 (Table 5). The anti-tumor activity of the combination was significantly greater than that of either single agent alone, and the EAR, indicating that the combination treatment effect was synergistic in this model. No significant animal weight loss was observed in any of the treatment groups over the course of treatment.

Collectively, these studies show that the dual agent treatment combination of KRas G12Ci and the SHP2 inhibitor RMC-4550 demonstrated significantly higher anti-tumor activity than that of either single agent in multiple KRAS G12C-driven tumor xenograft models. The dual agent combination treatment demonstrated additive effects in one NSCLC xenograft models (H1373) and one CRC xenograft model (SW837), and a synergistic effect in the NSCLC H358 xenograft model and the EL3187 PDX model. In addition, except for the one death reported early after combination treatment initiation in the H358 model, the combination treatment was well-tolerated across the models, suggesting that it may represent a safe and efficacious therapeutic for the treatment of cancer patients in the clinic.

As shown in Table 6, KRas G12Ci, TNO155, and the combination of KRas G12Ci and TNO155 were investigated in a panel of cancer cell lines with KRAS G12C mutation. Each cell line used for the studies was seeded in 384-well plate a day before adding treatment. Treatment time was 72 hours. After treatment time is met, 50 ul of CellTiter Glo was added to each well. After a 15 minute wait, plates were read using EnVision. The resulting data was used to calculate Ab1 IC50.

TABLE 6
KRas G12Ci and SHP2 (TNO155) Combination data

| | KRAS | SHP2 | | | | | | |
|---|---|---|---|---|---|---|---|---|
| | G12Ci | IC50 | Combo | Combination | | | | |
| Cell | IC50 | TNO155 | IC50 | Index | Biological | | G12C | DT |
| Line | (nM) | (nM) | (nM) | (CI) | Interpretation | Tissue | genotype | (h) |
| HCC44 | 10,000 | 48.46 | 12.48 | 0.0164 | synergistic | Lung | +/+ | 30 |
| SW756 | 6,941 | 16,218 | 72.37 | 0.0399 | synergistic | Cervix | +/− | 36 |
| NCI-H23 | 16.19 | 253.6 | 5.366 | 0.2981 | synergistic | Lung | +/− | 38 |
| NCI-H1373 | 5.28 | 156.2 | 3.728 | 0.6445 | additive | Lung | +/+ | 37 |
| EI-3187 | 0.3312 | 219.1 | 0.6669 | 0.6701 | additive | Lung | +/+ | 80 |
| NCI-H358 | 0.8891 | 171 | 3.343 | 0.8402 | additive | Lung | +/− | 38 |
| LU99 | 5850 | 779.5 | 46,066 | 0.8425 | additive | Lung | +/− | 25 |
| NCI-H1792 | 3716 | 56,431 | 5523 | 1 | additive | Lung | +/− | 32 |
| SW1573 | 7433 | 164.1 | 10.43 | 1 | additive | Lung | +/+ | 23 |

The combination demonstrated robust synergy and potency. The combination treatment demonstrated additive effects in multiple cell lines (NCI-H1373, EI-3187, NCI-H358, LU99, NCI-H1792, and SW1573) and demonstrated synergistic effects in other cell lines (HCC44, SW756, and NCI-H23). Combinations of KRas G12Ci with each of TNO155 and RMC-4550 exhibited similar synergy and potency in in vitro studies.

What is claimed is:

1. A method of treating a patient for cancer, comprising administering to a patient in need thereof, an effective amount of a compound of the formula: 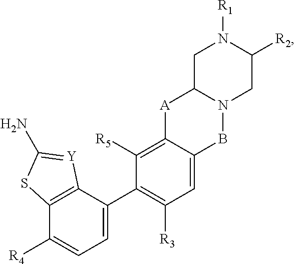
wherein:
A is —OCH₂—, —N(R₆)CH₂—, —OCH₂CH₂—, —N(R₆)CH₂CH₂—, —CH₂OCH₂—, or —CH₂N(R₆)CH₂—;
B is —CH₂— or —C(O)—;
Y is —C(CN)— or —N—;
R₁ is —CN, —C(O)C—CR₈, or a group of the formula 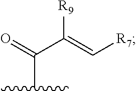
R₂ is H, methyl, or —CH₂CN;
R₃ and R₅ are each independently H, halogen, —C₀₋₃ alkyl-cyclopropyl, —C₁₋₆ alkyl optionally substituted 1-3 times with R₁₀, or —O—C₁₋₆ alkyl optionally substituted 1-3 times with R₁₀;
R₄ is H, halogen, or —C₁₋₆ alkyl optionally substituted 1-3 times with R₁₀;
R₆ is H or —C₁₋₆ alkyl optionally substituted 1-3 times with R₁₀;
R₇ is H, halogen, —NR₁₁R₁₂, —CH₂NR₁₁R₁₂, —C₁₋₆ alkyl optionally substituted 1-3 times with R₁₀ or R₁₃, —C₀₋₃ alkyl cyclopropyl, or —O—C₁₋₆ alkyl optionally substituted 1-3 times with R₁₀ or R₁₃;
R₈ is H, —C₁₋₄ alkyl optionally substituted 1-3 times with R₁₀, or —C₃₋₆ cycloalkyl optionally substituted 1-3 times with R₁₀;
R₉ is H, halogen, —CN, —C₀₋₃ alkyl-C₃₋₆ cycloalkyl, or —C₁₋₆ alkyl optionally substituted 1-3 times with R₁₀;
R₁₀ is independently at each occurrence halogen, oxygen, hydroxy, —C₁₋₄ alkyl, or —O—C₁₋₄ alkyl;
R₁₁ and R₁₂ are each independently H, —C₁₋₄ alkyl, or —C₁₋₄ heteroalkyl, wherein R₁₁ and R₁₂ may combine to form a heterocycloalkyl; and
R₁₃ is independently at each occurrence —N—C₁₋₄ alkyl,
or a pharmaceutically acceptable salt thereof; and
an effective amount of a SHP2 inhibitor, or a pharmaceutically acceptable salt thereof.

2. (canceled)

5. The method according to claim 1, wherein A is —OCH₂CH₂—, or a pharmaceutically acceptable salt thereof.

6. The method according to claim 1, wherein B is —C(O)—, or a pharmaceutically acceptable salt thereof.

7. The method according to claim 1, wherein Y is —C(CN)—, or a pharmaceutically acceptable salt thereof.

8. The method according to claim 1, wherein Y is —N—, or a pharmaceutically acceptable salt thereof.

9. The method according to claim 1, wherein R₁ is a group of the formula 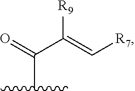
and wherein R₇ is H, F, Cl, methyl, ethoxy, ethyl, isopropyl, or cyclopropyl, or a pharmaceutically acceptable salt thereof.

10. The method according to claim 1, wherein R₁ is a group of the formula 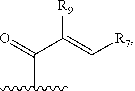
and wherein R₉ is H, F, Cl, —CHF₂, —CF₃, or —CH₂OH, or a pharmaceutically acceptable salt thereof.

11. The method according to claim 1, wherein R₁ is —CN,
—C(O)C≡CR₈, or a pharmaceutically acceptable salt thereof.

12. The method according to claim 1, wherein R₂ is H or methyl, or a pharmaceutically acceptable salt thereof.

13. The method according to claim 1, wherein R₃ is H, F, Cl, methyl, methoxy, ethyl, isopropyl, or cyclopropyl, or a pharmaceutically acceptable salt thereof.

14. The method according to claim 1, wherein R₄ is H, F, or Cl, or a pharmaceutically acceptable salt thereof.

15. The method according to claim 1, wherein R₅ is H, —CHF₂, —CH₂F, —CH₂OH, or —CH₂OCH₃, or a pharmaceutically acceptable salt thereof.

16. The method according to claim 1, wherein the compound is of the formula: 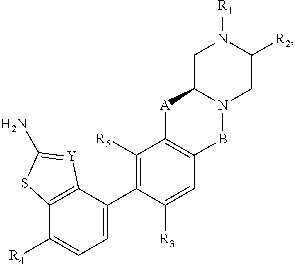
or a pharmaceutically acceptable salt thereof.

17. The method according to claim 1, wherein the compound is of the formula: 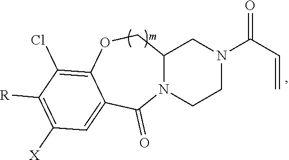
wherein R is 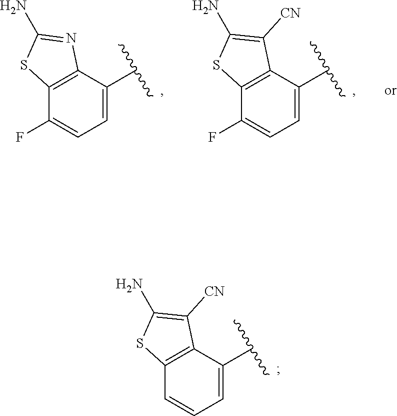 [SMILES: *c1ccc(F)c2sc(N)nc12 ; *c1ccc(F)c2sc(N)c(C#N)c12 ; *c1cccc2sc(N)c(C#N)c12]
X is Cl or F;
and m is 1 or 2,
or a pharmaceutically acceptable salt thereof.

18. The method according claim 1, wherein the compound is of the formula: 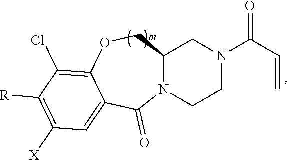
wherein R is 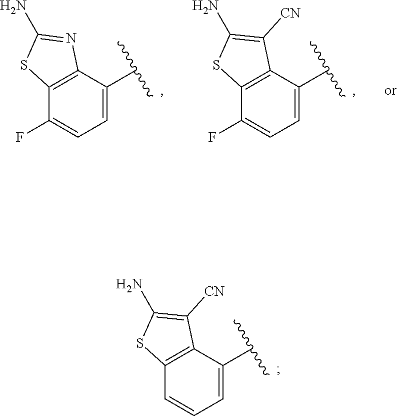 [SMILES: *c1ccc(F)c2sc(N)nc12 ; *c1ccc(F)c2sc(N)c(C#N)c12 ; *c1cccc2sc(N)c(C#N)c12]
X is Cl or F;
and m is 1 or 2,
or a pharmaceutically acceptable salt thereof.

19. The method according claim 1, wherein the compound is of the formula: 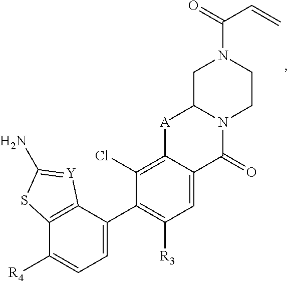
wherein:
A is —OCH₂— or —OCH₂CH₂—;
Y is C(CN) or N;
R₃ is Cl or F;
R₄ is H or F when Y is C(CN); and
R₄ is F when Y is N,
or a pharmaceutically acceptable salt thereof.

20. The method according claim 1, wherein A is

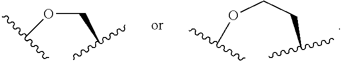

21. The method according claim 1, wherein the compound is: 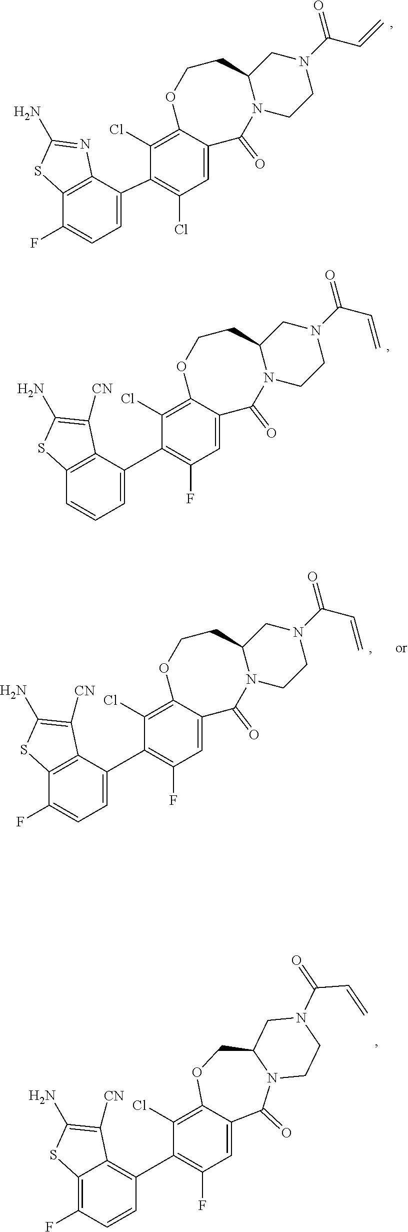 [SMILES: C=CC(=O)N1CCN2C(=O)c3cc(Cl)c(-c4ccc(F)c5sc(N)nc45)c(Cl)c3OCC[C@H]2C1 ; C=CC(=O)N1CCN2C(=O)c3cc(F)c(-c4cccc5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OC[C@H]2C1]
or a pharmaceutically acceptable salt thereof.

22. The method according claim 1, wherein the compound is:

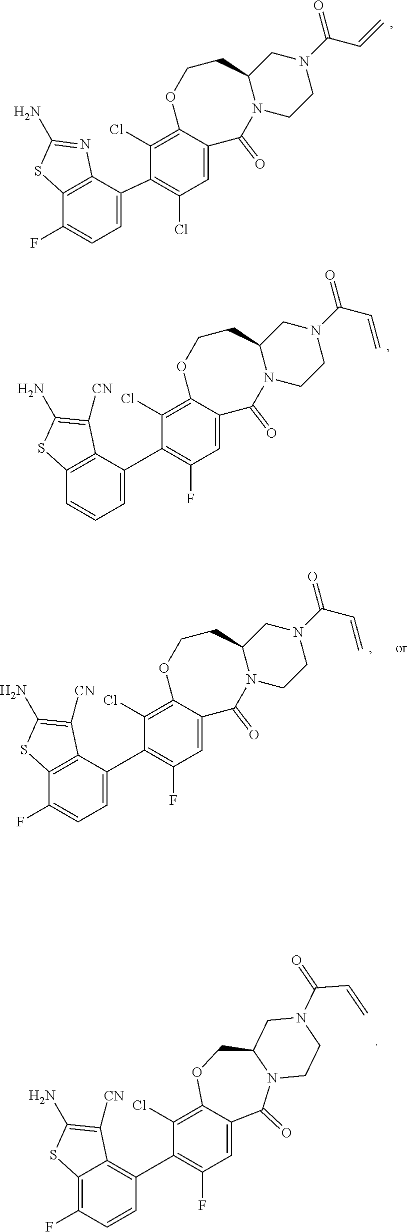 [SMILES: C=CC(=O)N1CCN2C(=O)c3cc(Cl)c(-c4ccc(F)c5sc(N)nc45)c(Cl)c3OCC[C@H]2C1 ; C=CC(=O)N1CCN2C(=O)c3cc(F)c(-c4cccc5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OCC[C@H]2C1 ; C=CC(=O)N1CCN2C(=O)c3cc(F)c(-c4ccc(F)c5sc(N)c(C#N)c45)c(Cl)c3OC[C@H]2C1]

23. The method according to claim 1, wherein the SIP2 inhibitor is selected from the group consisting of a Type I SHP2 inhibitor, a Type II SHP2 inhibitor, BBP-398, IACS-15509, or IACS-13909, X37, ERAS-601, SH3809, HBI-2376, ETS-001, or PCC0208023, or a pharmaceutically acceptable salt thereof.

24. The method according to claim 1, wherein the Type I SHP2 inhibitor is selected from the group consisting of PHPS1 or GS-493, NSC-87877 or NSC-117199, Cefsulodin, or a pharmaceutically acceptable salt thereof.

25. The method according to claim 1, wherein the Type II SHP2 inhibitor is selected from the group consisting of JAB-3068 or JAB-3312, RMC-4550 or RMC-4630, SHP099, SHP244, SHP389, SHP394, or TN0155, RG-6433 or RLY-1971, or a pharmaceutically acceptable salt thereof.

26. The method according to claim 1, wherein the Type II SHP2 inhibitor is selected from the group consisting of JAB-3068, RMC-4630, TN0155, or RLY-1971, or a pharmaceutically acceptable salt thereof.

27. A method according to claim 1, wherein the cancer is selected from the group consisting of lung cancer, pancreatic cancer, cervical cancer, esophageal cancer, endometrial cancer, ovarian cancer, cholangiocarcinoma, and colorectal cancer.

28. The method according to claim 1, wherein the cancer is non-small cell lung cancer, and wherein one or more cells express KRas G12C mutant protein with or without a SHP2 dysregulation or overexpression.

29. The method according to claim 1, wherein the cancer is colorectal cancer, and wherein one or more cells with or without a SHP2 dysregulation or overexpression express KRas G12C mutant protein.

30. The method according to claim 1, wherein the cancer is pancreatic cancer, and wherein one or more cells with or without a SHP2 dysregulation or overexpression express KRas G12C mutant protein.

31. The method according to claim 1, wherein the patient has a cancer that has a KRAS G12C mutation.

32. The method according to claim 1, wherein the patient has a cancer that was determined to have one or more cells expressing the KRas G12C mutant protein prior to administration of the compound, or a pharmaceutically acceptable salt thereof, or the SHP2 inhibitor, or a pharmaceutically acceptable salt thereof.

33. The method according to claim 1, wherein the compound of the formula and the SHP2 inhibitor are provided in simultaneous or sequential combination to the patient in need thereof.

34. The method according to claim 1, wherein the compound of the formula and the SHP2 inhibitor are provided in simultaneous combination to the patient in need thereof.

35. The method according to claim 1, wherein the compound of the formula and the SHP2 inhibitor are provided in sequential combination to the patient in need thereof.

36. The method according to claim 1, wherein the compound of the formula is provided to the patient in need thereof before the SHP2 inhibitor is provided to the patient in need thereof.

37. The method according to claim 1, wherein the SHP2 inhibitor is provided to the patient in need thereof before the compound of the formula is provided to the patient in need thereof.
